SEQUENCE LISTING (31 sequences)

SEQ ID NO: 1 - protein, 490 aa, Homo sapiens
MKDPPDLLDRQKCPNALASLRHAKWFQARANGLKSCVIVLRILRDLCNRVPTWAPLKGWPLELICEKSIGTCNRPLGAGEALRRVMECLASGILLPGGPGLHDPCERDPTDALSYMTIQQKEDITHSAQHALRLSAFGQIYKVLEMDPLPSSKPFQKYSWSVTDKEGAGSSALKRPFEDGLGDDKDPNKKMKRNLRKILDSKAIDLMNALMRLNQIRPGLQYKLLSQSGPVHAPVFTMSVDVDGTTYEASGPSKKTAKLHVAVKVLQAMGYPTGFDADIECMSSDEKSDNESKNETVSSNSSNNTGNSTTETSSTLEVRTQGPILTASGKNPVMELNEKRRGLKYELISETGGSHDKRFVMEVEVDGQKFRGAGPNKKVAKASAALAALEKLFSGPNAANNKKKKIIPQAKGVVNTAVSAAVQAVRGRGRGTLTRGAFVGATAAPGYIAPGYGTPYGYSTAAPAYGLPKRMVLLPVMKFPTYPVPHYSFF

SEQ ID NO: 2 - protein, 366 aa, Homo sapiens
MSEEEQGSGTTTGCGLPSIEQMLAANPGKTPISLLQEYGTRIGKTPVYDLLKAEGQAHQPNFTFRVTVGDTSCTGQGPSKKAAKHKAAEVALKHLKGGSMLEPALEDSSSFSPLDSSLPEDIPVFTAAAAATPVPSVVLTRSPPMELQPPVSPQQSECNPVGALQELVVQKGWRLPEYTVTQESGPAHRKEFTMTCRVERFIEIGSGTSKKLAKRNAAAKMLLRVHTVPLDARDGNEVEPDDDHFSIGVGFRLDGLRNRGPGCTWDSLRNSVGEKILSLRSCSLGSLGALGPACCRVLSELSEEQAFHVSYLDIEELSLSGLCQCLVELSTQPATVCHGSATTREAARGEAARRALQYLKIMAGSK

SEQ ID NO: 3 - protein, 803 aa, Homo sapiens
MRALWVLGLCCVLLTFGSVRADDEVDVDGTVEEDLGKSREGSRTDDEVVQREEEAIQLDGLNASQIRELREKSEKFAFQAEVNRMMKLIINSLYKNKEIFLRELISNASDALDKIRLISLTDENALSGNEELTVKIKCDKEKNLLHVTDTGVGMTREELVKNLGTIAKSGTSEFLNKMTEAQEDGQSTSELIGQFGVGFYSAFLVADKVIVTSKHNNDTQHIWESDSNEFSVIADPRGNTLGRGTTITLVLKEEASDYLELDTIKNLVKKYSQFINFPIYVWSSKTETVEEPMEEEEAAKEEKEESDDEAAVEEEEEEKKPKTKKVEKTVWDWELMNDIKPIWQRPSKEVEEDEYKAFYKSFSKESDDPMAYIHFTAEGEVTFKSILFVPTSAPRGLFDEYGSKKSDYIKLYVRRVFITDDFHDMMPKYLNFVKGVVDSDDLPLNVSRETLQQHKLLKVIRKKLVRKTLDMIKKIADDKYNDTFWKEFGTNIKLGVIEDHSNRTRLAKLLRFQSSHHPTDITSLDQYVERMKEKQDKIYFMAGSSRKEAESSPFVERLLKKGYEVIYLTEPVDEYCIQALPEFDGKRFQNVAKEGVKFDESEKTKESREAVEKEFEPLLNWMKDKALKDKIEKAVVSQRLTESPCALVASQYGWSGNMERIMKAQAYQTGKDISTNYYASQKKTFEINPRHPLIRDMLRRIKEDEDDKTVLDLAVVLFETATLRSGYLLPDTKAYGDRIERMLRLSLNIDPDAKVEEEPEEEPEETAEDTTEDTEQDEDEEMDVGTDEEEETAKESTAEKDEL

SEQ ID NO: 4 - protein, 181 aa, Homo sapiens
MAGSREVVAMDCEMVGLGPHRESGLARCSLVNVHGAVLYDKFIRPEGEITDYRTRVSGVTPQHMVGATPFAVARLEILQLLKGKLVVGHDLKHDFQALKEDMSGYTIYDTSTDRLLWREAKLDHCRRVSLRVLSERLLHKSIQNSLLGHSSVEDARATMELYQISQRIRARRGLPRLAVSD

SEQ ID NO: 5 - protein, 658 aa, Homo sapiens
MVKHSTIYPSPEELEAVQNMVSTVECALKHVSDWLDETNKGTKTEGETEVKKDEAGENYSKDQGGRTLCGVMRIGLVAKGLLIKDDMDLELVLMCKDKPTETLLNTVKDNLPIQIQKLTEEKYQVEQCVNEASIIIRNTKEPTLTLKVILTSPLIRDELEKKDGENVSMKDPPDLLDRQKCLNALASLRHAKWFQARANGLKSCVIVLRILRDLCNRVPTWAPLKGWPLELICEKSIGTCNRPLGAGEALRRVMECLASGILLPGGPGLHDPCERDPTDALSYMTIQQKEDITHSAQHALRLSAFGQIYKVLEMDPLPSSKPFQKYSWSVTDKEGAGSSALKRPFEDGLGDDKDPNKKMKRNLRKILDSKAIDLMNALMRLNQIRPGLQYKLLSQSGPVHAPVFTMSVDVDGTTYEASGPSKKTAKLHVAVKVLQAMGYPTGFDADIECMSSDEKSDNESKNETVSSNSSNNTGNSTTETSSTLEVRTQGPILTASGKNPVMELNEKRRGLKYELISETGGSHDKRFVMEVEVDGQKFRGAGPNKKVAKASAALAALEKLFSGPNAANNKKKKIIPQAKGVVNTAVSAAVQAVRGRGRGTLTRGAFVGATAAPGYIAPGYGTPYGYSTAAPAYGLPKRMVLLPVMKFPTYPVPHYSFF

SEQ ID NO: 6 - protein, 405 aa, Homo sapiens
MECLASGILLPGGPGLHDPCERDPTDALSYMTIQQKEDITHSAQHALRLSAFGQIYKVLEMDPLPSSKPFQKYSWSVTDKEGAGSSALKRPFEDGLGDDKDPNKKMKRNLRKILDSKAIDLMNALMRLNQIRPGLQYKLLSQSGPVHAPVFTMSVDVDGTTYEASGPSKKTAKLHVAVKVLQAMGYPTGFDADIECMSSDEKSDNESKNETVSSNSSNNTGNSTTETSSTLEVRTQGPILTASGKNPVMELNEKRRGLKYELISETGGSHDKRFVMEVEVDGQKFRGAGPNKKVAKASAALAALEKLFSGPNAANNKKKKIIPQAKGVVNTAVSAAVQAVRGRGRGTLTRGAFVGATAAPGYIAPGYGTPYGYSTAAPAYGLPKRMVLLPVMKFPTYPVPHYSFF

SEQ ID NO: 7 - protein, 702 aa, Homo sapiens [sequence omitted]

SEQ ID NO: 8 - protein, 764 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAEQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSQFSRPPPPSRPRCCVVRCSGSPCGPSCDPYLAVFGTPCLQWFVSCHYNFVWVEFLSFCSSVSLCLFTLRVSGNSVCL

SEQ ID NO: 9 - protein, 702 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAEQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSDFFTDCYGYHDFGSS

SEQ ID NO: 10 - protein, 690 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAEQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYTGFV

SEQ ID NO: 11 - protein, 894 aa, Homo sapiens [sequence omitted]

SEQ ID NO: 12 - protein, 564 aa, Homo sapiens
QLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAENVKQQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSKCAFLSV

SEQ ID NO: 13 - protein, 671 aa, Homo sapiens
MRPMRIFVNDDRHVMAKHSSVYPTQEELEAVQNMVSHTERALKAVSDWIHEQEKGSSEQAESDNMDVPPEDDSKEGAGEQKTEHMTRTCRGVMRAGPGGQSASYSRGTWIWSWCCCVRRSPQPALLDKVADNLAIQLAAVTEDKYEILQSVDDAAIVIKNTKEPPLSLTIHLTSPVVREEMEKVLAGETLSVNDPPDVLDRQKCFAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAENVKQQGPILTKHGKNPVMELNEKRRGLKYELISETGGSHDKRFVMEVEVDGQKFQGAGSNKKVAKAYAALAALEKLFPDTPLSPLMPTKRREPQYPSEGDRNLLLSHITLASAWEAPCTTKCPHPPTFEGGEEAGRSGDEGAGEDLVAPTMEAT

SEQ ID NO: 14 - protein, 611 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIVTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAEQGPILTKHGKNPVMELNEKRRGLKYELISETGGSHDKRFVMEVEVDGQKFQGAGSNKKVAKAYAALAALEKLFPDTPLALDANKKKRAPVPVRGG

SEQ ID NO: 15 - protein, 702 aa, Homo sapiens [sequence omitted]

SEQ ID NO: 16 - protein, 440 aa, Homo sapiens
MRPMRIFVNDDRHVMAKHSSVYPTQEELEAVQNMVSHTERALKAVSDWIDEQEKGSSEQAESDNMDVPPEDDSKEGAGEQKTEHMTRTLRGVMRVGLVAKGLLLKGDLDLELVLLCKEKPTTALLDKVADNLAIQLAAVTEDKYEILQSVDDAAIVIKNTKEPPLSLTIHLTSPVVREEMEKVLAGETLSVNDPPDVLDRQKCLAALASLRHAKWFQARANGLKSCVIVIRVLRDLCTRVPTWGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDG

SEQ ID NO: 17 - protein, 345 aa, Homo sapiens [sequence omitted]

SEQ ID NO: 18 - protein, 313 aa, Homo sapiens
MSQSRHRAEAPPLEREDSGTFSLGKMITAKPGKTPIQVLHEYGMKTKNIPVYECERSDVQIHVPTFTFRVTVGDITCTGEGTSKKLAKHRAAEAAINILKANASICFAVPDPLMPDPSKQPKNQLNPIGSLQELAIHHGWRLPEYTLSQEGGPAHKREYTTICRLESFMETGKGASKKQAKRNAAEKFLAKFSNISPENHISLTNVVGHSLGCTWHSLRNSPGEKINLLKRSLLSIPNTDYIQLLSEIAKEQGFNITYLDIDELSANGQYQCLAELSTSPITVCHGSGISCGNAQSDAAHNALQYLKIIAERK

SEQ ID NO: 19 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 20 - protein, 1225 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETRRNAEFLTCNIPTSNASNNMVTTEKVENGQEPVKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 21 - protein, 702 aa, Homo sapiens; identical to SEQ ID NO: 9

SEQ ID NO: 22 - protein, 764 aa, Homo sapiens; identical to SEQ ID NO: 8

SEQ ID NO: 23 - protein, 591 aa, Homo sapiens
GHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAEQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSQFYSNGGHSGNASGGGGGGGGGSSGYGSYYQGDNYNSPVPPKHAGKKQPHGGQQKPSYGSGYQSHQGQQQSYNQSLYSNYGPPQGKQKGYNHGQGSYSYSNSYNSPGGGGGSDYNYESKFNYSGSGGRSGGNSYGSGGASYNPGSHGGYGGGSGGGSSYQGKQGGYSQSNYNSPGSGQNYSGPPSSYQSSQGGYGRNADHSMNYQYR

SEQ ID NO: 24 - protein, 514 aa, Homo sapiens
TLSVNDPPDVLDRQKCLAALASLRHAKWFQARANGLKSCVIVIRVLRDLCTRVPTGPLRGWPLELLCEKSIGTANRPMGAGEALRRVLECLASGIVMPDGSGIYDPCEKEATDAIGHLDRQQREDITQSAQHALRLAAFGQLHKVLGMDPLPSKMPKKPKNENPVDYTVQIPPSTTYAITPMKRPMEEDGEEKSPSKKKKKIQKKEEKAEPPQAMNALMRLNQLKPGLQYKLVSQTGPVHAPIFTMSVEVDGNSFEASGPSKKTAKLHVAVKVLQDMGLPTGAEGRDSSKGEDSAEETEAKPAVVAPAPVVEAVSTPSAAFPSDATAEQGPILTKHGKNPVMELNEKRRGLKYELISETGGSHDKRFVMEVEVDGQKFQGAGSNKKVAKAYAALAALEKLFPDTPLALDANKKKRAPVPVRGGPKFAAKPHNPGFGMGGPMHNEVPPPPNLRGRGRGGSIRGRGRGRGFGGANHGGYMNAGAGYGSYGYGGNSATAGYSDFFTDCYGYHDFGSS

SEQ ID NO: 25 - protein, 422 aa, Homo sapiens
MGKAKVPASKRAPSSPVAKPGPVKTLTRKKNKKKKRFWKSKAREVSKKPASGPGAVVRPPKAPEDFSQNWKALQEWLLKQKSQAPEKPLVISQMGSKKKPKIIQQNKKETSPQVKGEEMPAGKDQEASRGSVPSGSKMDRRAPVPRTKASGTEHNKKGTKERTNGDIVPERGDIEHKKRKAKEAAPAPPTEEDIWFDDVDPADIEAAIGPEAAKIARKQLGQSEGSVSLSLVKEQAFGGLTRALALDCEMVGVGPKGEESMAARVSIVNQYGKCVYDKYVKPTEPVTDYRTAVSGIRPENLKQGEELEVVQKEVAEMLKGRILVGHALHNDLKVLFLDHPKKKIRDTQKYKPFKSQVKSGRPSLRLLSEKILGLQVQQAEHCSIQDAQAAMRLYVMVKKEWESMARDRRPLLTAPDHCSDDA

SEQ ID NO: 26 - protein, 422 aa, Homo sapiens
MGKAKVPASKRAPSSPVAKPGPVKTLTRKKNKKKKRFWKSKAREVSKKPASGPGAVVRPPKAPEDFSQNWKALQEWLLKQKSQAPEKPLVISQMGSKKKPKIIQQNKKETSPQVKGEEMPAGKDQEASRGSVPSGSKMDRRAPVPRTKASGTEHNKKGTKERTNGDIVPERGDIEHKKRKAKEAAPAPPTEEDIWFDDVDPPDIEAAIGPEAAKIARKQLGQSEGSVSLSLVKEQAFGGLTRALALDCEMVGVGPKGEESMAARVSIVNQYGKCVYDKYVKPTEPVTDYRTAVSGIRPENLKQGEELEVVQKEVAEMLKGRILVGHALHNDLKVLFLDHPKKKIRDTQKYKPFKSQVKSGRPSLRLLSEKILGLQVQQAEHCSIQDAQAAMRLYVMVKKEWESMARDRRPLLTAPDHCSDDA

SEQ ID NO: 27 - protein, 422 aa, Homo sapiens; identical to SEQ ID NO: 26

SEQ ID NO: 28 - protein, 422 aa, Homo sapiens; identical to SEQ ID NO: 25

SEQ ID NO: 29 - protein, 353 aa, Homo sapiens
MSTLLLNLDFGEPPPKKALEGNAKHRNFVKKRRLLERRGFLSKKNQPPSKAPKLHSEPSKKGETPTVDGTWKTPSFPKKKTAASSNGSGQPLDKKAAVSWLTPAPSKKADSVAAKVDLLGEFQSALPKINSHPTRSQKKSSQKKSSKKNHPQKNAPQNSTQAHSENKCSGASQKLPRKMVAIDCEMVGTGPKGHVSSLARCSIVNYNGDVLYDEYILPPCHIVDYRTRWSGIRKQHMVNATPFKIARGQILKILTGKIVVGHAIHNDFKALQYFHPKSLTRDTSHIPPLNRKADCPENATMSLKHLTKKLLNRDIQVGKSGHSSVEDAQATMELYKLVEVEWEEHLARNPPTD

SEQ ID NO: 30 - protein, 208 aa, Homo sapiens
MVGTGPRGRVSELARCSIVSYHGDVLYDKYIRPEMPIADYRTRWSGITRQHMRKAVPFQVAQKEILKLLKGKVVVGHALHNDFQALKYVHPRSQTRDTTYVPNFLSEPGLHTRARVSLKDLALQLLHKKIQVGQHGHSSVEDATTAMELYRLVEVQWEQQEARSLWTCPEDREPDSSTDMEQYMEDQYWPDDLAHGSRGGAREAQDRR

SEQ ID NO: 31 - DNA, 476 bp, Homo sapiens
  misc_feature (1)..(476): n = a, c, t, g, any, unknown, or other
tctggctcacctggaggaagaggtccacgacaaggtggttgcccgggacctggttggagccatgttgagcccactgccgcagccacgcccctctgccccccaggtgctggcccaccccgttcttttggagcagancaagcaactccagttcttccaggacgtcagtgactggctggagaaggagtccgagcaggagcccctggtgagggcactggaggcgggaggctgcgcagtggtccgggacaactggcacgagcacatctccatgccgctgcagacagatctgagaaagttccggtcctataaggggacatcagtgcgagacctgctccgtgctgtgaggaacaagaagcaccactacagggagctcccagttgaggtgcgacaggcattcggccaagtccctgatggcttcgtccagtacttcacaaaccgctttccaanggctgctcntcaaangcaccgagccatgagga

(19) United States
(12) Patent Application Publication
(10) Pub. No.: US 2003/0125285 A1
(43) Pub. Date: Jul. 3, 2003

(54) Method of examining efficacy of therapy with nucleic acid

(76) Inventors: Kazuko Hirabayashi, Kyoto-shi (JP); Junichi Yano, Nara-shi (JP)

Correspondence Address: BIRCH STEWART KOLASCH & BIRCH, PO BOX 747, FALLS CHURCH, VA, 22040-0747, US

(21) Appl. No.: 10/233,553

(22) Filed: Sep. 4, 2002

(30) Foreign Application Priority Data
Sep. 4, 2001 (JP) 2001-267385

(51) Int. Cl. A61K 48/00; C12Q 1/68; G01N 33/53; A61K 31/7076; A61K 31/7072

(52) U.S. Cl. 514/44; 514/45; 514/49; 435/6; 435/7.1

(57) ABSTRACT

The present invention provides a method of examining efficacy of treatment with a nucleotide or a nucleoside having antitumor- or antivirus activity in individual patients beforehand, which method is mainly connected to tailor-made therapy, a method of screening for agents potentially having antitumor or antivirus activity, and method of investigating the mechanism of action of an antitumor or antivirus agent in vivo.

### TECHNICAL FIELD

The present invention is related to a method of examining efficacy of treatment with an antitumor- or antivirus agent in individual patients. The present invention is also related to a method of screening for a candidate antitumor or antivirus agent.

### BACKGROUND OF THE INVENTION

For example, synthetic nucleic acid polymers typified by polyinosinic—polycytidylic acid (i.e., polyinosinic acid•polycytidylic acid) have excellent antitumor and/or antivirus activity. Such a synthetic nucleic acid polymer has been reported that, when administered into a living body as a complex with an appropriate cationic liposome carrier, it attacks specifically cancer cells and activates an intracellular nuclease thereby leads the cells into apoptosis and that it has prominent antivirus effect on hepatitis virus (WO99/20283, WO99/48531)

However, the reactivity to antitumor agent administered differs from one patient to another, irrespective of the high potency of the drug administered, and an agent having sufficient effects in a patient while shows only poor effect in another.

Recently, tailor-made therapy, which is a method of treatment adapted to individual patients, has been proposed. The tailor-made therapy wherein treatment is conducted while confirming the efficacy or predicting side effects of a drug is a promising new therapeutic system.

### SUMMARY OF THE INVENTION

The present invention provides a method of examining efficacy (effectiveness) of treatment with antitumor- or antivirus agent in individual patients, which method is mainly connected to tailor-made therapy. The present invention also provides a method of screening for agents potentially having antitumor or antivirus activity.

The present inventors have intensively studied and found that there exist in a living body proteins capable of binding to or induced to express by a double-stranded RNA, which is polyinosinic—polycytidylic acid known to be an excellent antitumor or antivirus agent, and established the present invention. In the present application, three proteins of the former type and one protein of the latter type will be disclosed.

The present invention, for example, includes the following embodiments.

(1) A method of examining efficacy of treatment with a nucleotide or a nucleoside in a given subject comprising:

(a) measuring the amount of expression or activity of at least one protein among those each having amino acid sequences of SEQ ID NO: 1-4 in a blood or tissue sample previously taken from the subject;

(b) administering the nucleotide or the nucleoside into a tissue of the subject or a tissue taken from the subject followed by measuring the amount of expression or activity of the same protein as that measured in (a) in blood or treated tissue after 3-48 hours from administration; and

(c) comparing the measurement obtained in step (a) with that obtained in step (b) and evaluating the change in the amount of expression or the activity of the protein.

(2) A method of examining efficacy of treatment with a nucleotide or a nucleoside in a given subject comprising:

(a) measuring the amount of expression or activity of at least one protein among those each having amino acid sequences of SEQ ID NO: 5-30 in a blood or tissue sample previously taken from the subject;

(b) administering the nucleotide or the nucleoside into a tissue of the subject or a tissue taken from the subject followed by measuring the amount of expression or activity of the same protein as that measured in (a) in blood or treated tissue after 3-48 hours from administration; and

(c) comparing the measurement obtained in step (a) with that obtained in step (b) and evaluating the change in the amount of expression or the activity of the protein.

(3) A method of screening for agents potentially having antitumor or antivirus activity comprising examining a test compound for ability to bind to at least one protein among those each having amino acid sequences of SEQ ID NO: 1-4.

(4) A method of screening for agents potentially having antitumor or antivirus activity comprising examining a test compound for ability to bind to at least one protein among those each having amino acid sequences of SEQ ID NO: 5-30.

(5) A method of investigating the mechanism of action of an antitumor or antivirus agent in vivo comprising examining the antitumor or antivirus agent for ability to bind to at least one protein among those each having amino acid sequences of SEQ ID NO: 1-4.

(6) A method of investigating the mechanism of action of an antitumor or antivirus agent in vivo comprising examining the antitumor or antivirus agent for ability to bind to at least one protein among those each having amino acid sequences of SEQ ID NO: 5-30.

### BRIEF DESCRIPTION OF THE DRAWINGS

FIG. 1 is a schematic diagram of the process for detecting proteins each having amino acid sequences of SEQ ID NO: 1-3 described in Experiment 1.

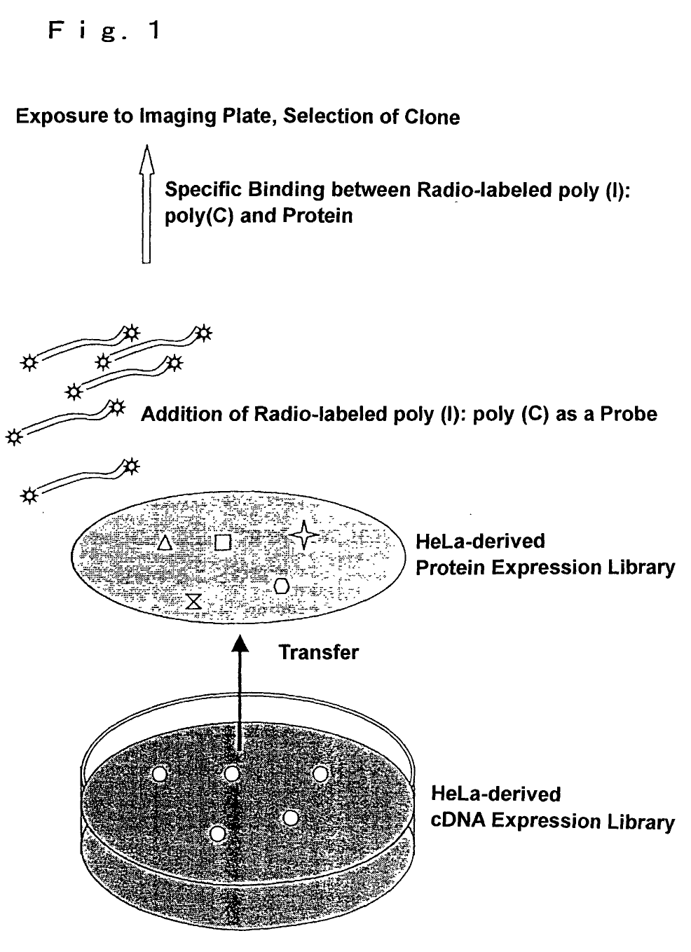

FIG. 2 is an electrophoretogram showing expression of mRNA for ISG20 in carcinoma cells, HeLaS3 (left lane) and HeLa (right lane) following the addition of a complex comprising polyinosinic—polycytidylic acid. The figures at the upper side indicate the time (hour) after administration of the complex. The bands at the rightmost lane indicate markers. The white arrow indicates the site where mRNA for ISG20 is expressed.

FIG. 3 is an electrophoretogram showing expression of mRNA for ISG20 in carcinoma cells, A431 (left lane) and SW620 (right lane) following the addition of a complex comprising polyinosinic—polycytidylic acid. The figures at the upper side indicate the time (hour) after administration of the complex. The bands at the middle lane indicate markers. The white arrow indicates the site where mRNA for ISG20 is expressed.

### DETAILED DESCRIPTION OF THE PREFERRED EMBODIMENTS

Nucleotides or nucleosides of which antitumor or antivirus activity is evaluated by the present method are those that exert activity when entered into a cell.

Such a nucleotide or nucleoside is, for example, administered into a tissue as an aqueous solution containing the same as it is, or as a pharmaceutical composition containing the same in association with an appropriate carrier. The proteins usable in the present method can be obtained, for example, according to the screening method illustrated hereinafter in the Experimental Examples.

I. Proteins

Examples of proteins usable in the present method include those each having amino acid sequences of SEQ ID NO: 1-30.

The proteins having amino acid sequences of SEQ ID NO: 1-4 are all known proteins.

The protein of SEQ ID NO: 1 exists in a living body and comprises 490 amino acids. A gene encoding the protein is deposited at GenBank under accession number AK002169 (gene name: AK002169). This protein is known to comprise motifs of ribonuclease III family, receptor tyrosine kinase class III, receptor tyrosine kinase class V, dsRNA binding motif, and the like. Since the motifs of nuclease and ribonuclease exist, the protein is considered to induce apoptosis in cells when activated.

The proteins of SEQ ID NO: 5-16 are all known proteins, of which amino acid sequences are highly identical with the amino acid sequence of the protein of SEQ ID NO: 1 wherein the sequence identity is 60% or more, and considered to have activity equivalent to that of the protein of SEQ ID NO: 1. Genes encoding these proteins are deposited at GenBank, wherein the GenBank accession number of a gene encoding the protein of SEQ ID NO: 5 is AL136866 (gene name; HSM801834), the GenBank accession number of a gene encoding the protein of SEQ ID NO: 6 is AK024285 (gene name; AK024285), the GenBank accession number of a gene encoding the protein of SEQ ID NO: 7 is AF167569 (gene name; AF167569), the GenBank accession number of a gene encoding the protein of SEQ ID NO: 8 is AF141870 (gene name; AF141870), the GenBank accession number of a gene encoding the protein of SEQ ID NO: 9 is AJ271745 (gene name; HSA271745), the GenBank accession number of a gene encoding the protein of SEQ ID NO: 10 is AJ271746 (gene name; HSA271746), the GenBank accession number of a gene encoding the protein of SEQ ID NO: 11 is AF167570 (gene name; AF167570), the GenBank accession number of a gene encoding the protein of SEQ ID NO: 12 is AJ271744 (gene name; HSA271744), the GenBank accession number of a gene encoding the protein of SEQ ID NO: 13 is U10324 (gene name; HSU10324), the GenBank accession number of a gene encoding the protein of SEQ ID NO: 14 is X98265 (gene name; HSMPP4II), the GenBank accession number of a gene encoding the protein of SEQ ID NO: 15 is AF147209 (gene name; AF147209), and the GenBank accession number of a gene encoding the protein of SEQ ID NO: 16 is X98264 or X98265 (gene name; HSMPP4I).

The protein having amino acid sequence of SEQ ID NO: 2 exists in a living body and comprises 366 amino acids and is referred to as human TAR RNA binding protein 2 (TRBP2). A gene encoding the protein is deposited at GenBank under accession number U08998 (gene name: HSU08998). This protein is also known to have motifs such as a motif of ribonuclease III family, dsRNA binding motif, and the like. Since the motifs of nuclease and kinase exist, the protein is considered to induce apoptosis in cells when activated.

The proteins of SEQ ID NO: 17-24 are all known proteins, which comprise amino acid sequences having high sequence identity to the amino acid sequence of dsRNA binding motif portion of the protein of SEQ ID NO: 2, and are considered to have activity equivalent to that of the protein of SEQ ID NO: 2. Genes encoding these proteins are deposited at GenBank, wherein the GenBank accession number of a gene encoding the protein of SEQ ID NO: 17 is M60801 (gene name; HUMTRBP), the GenBank accession number of a gene encoding the protein of SEQ ID NO: 18 is AF083033 (gene name; AF083033), the GenBank accession number of a gene encoding the protein of SEQ ID NO: 19 is X99227 (gene name; HSHRED1A), the GenBank accession number of a gene encoding the protein of SEQ ID NO: 20 is U18121 (gene name; HSU18121), the GenBank accession number of a gene encoding the protein of SEQ ID NO: 21 is AJ271745 (gene name; HSA271745), the GenBank accession number of a gene encoding the protein of SEQ ID NO: 22 is AF141870 (gene name; AF141870), the GenBank accession number of a gene encoding the protein of SEQ ID NO: 23 is AJ271747 (gene name; HSA271747), the GenBank accession number of a gene encoding the protein defined by SEQ ID NO: 24 is AF202445 (gene name; AF202445),

The protein having amino acid sequence of SEQ ID NO: 3 exists in a living body, comprises 803 amino acids, and is referred to as human TRAL. A gene encoding the protein is deposited at GenBank under accession number X15187 (gene name: HSTRA1). This protein is presumed to participate in the transmission of a signal for cell growth or cell death in association with other protein(s) (Saishin Igaku, vol. 56, p. 21, 2001). Examples of the other proteins include IRE1 β having nuclease motif (e.g., Nature Cell Biology, vol. 3, p. 158, 2001). It is considered that, when the protein of SEQ ID NO: 3 is activated, the activity of IRE1 β is controlled and apoptosis in a cell is induced. A partial sequence of a DNA encoding IRE1 β is deposited at GenBank under accession number AA088547 (protein name: IRE1 β) (SEQ ID NO: 31).

The protein having amino acid sequence of SEQ ID NO: 4 exists in a living body, comprises 181 amino acids and is also referred to as ISG20. A gene encoding the protein is deposited at GenBank under accession number X89773 (gene name: HSISG20GN). This protein is reported to have the both of ribonuclease activity (RNase) and deoxyribonuclease activity (DNase) (e.g., Biochemistry, 40(24), p.7174, 2001). The present inventors have found that, when a complex comprising polyinosinic—polycytidylic acid double stranded RNA and cationic liposomes is added to a certain cancer cell, mRNA for said protein is expressed, which cationic liposomes are formed from 2-O-(2-diethylaminoethyl)carbamoyl-1,3-O-dioleoyl glycerol (hereinafter, referred to as “cationic glycerol”) and a phospholipid as essential components, as shown in Experimental Example 2 below.

The proteins of SEQ ID NO: 25-30 are all known proteins, which comprise amino acid sequences having high sequence identity to the amino acid sequence of nuclease motif of the protein of SEQ ID NO: 4. Genes encoding these proteins are deposited at GenBank, wherein the GenBank accession number of a gene encoding the protein of SEQ ID NO: 25 is AK025493 (gene name; AK025493), the GenBank accession number of a gene encoding the protein of SEQ ID NO: 26 is AF273304 (gene name; AF273304), the GenBank accession number of a gene encoding the protein of SEQ ID NO: 27 is AF295774 (gene name; AF295774), the GenBank accession number of a gene encoding the protein of SEQ ID NO: 28 is AL136894 (gene name; HSM801862), the GenBank accession number of a gene encoding the protein of SEQ ID NO: 29 is AK022733 (gene name; AK022733), the GenBank accession number of a gene encoding the protein of SEQ ID NO: 30 is AK022546 (gene name; AK022546).

II. Nucleotide or Nucleoside, or Composition Containing the Same

The nucleotides or nucleosides usable in the present method are not particularly restricted as long as they can exert antitumor or antivirus activity when entered into a cell. There are mono-, oligo- and poly-nucleotides, and the present invention encompasses all of them without any particular limitations.

Examples of the nucleotides or nucleoside are shown below.

(1) Homopolymer•homopolymer Double-stranded RNA

Polyinosinic acid•polycytidylic acid,

Polyinosinic acid•poly(5-bromocytidylic acid),

Polyinosinic acid•poly (2-thiocytidylic acid),

Poly(7-deazainosinic acid)•polycytidylic acid,

Poly(7-deazainosinic acid)•poly(5-bromocytidylic acid),

Poly(2′-azidoinosinic acid)•polycytidylic acid,

Polyinosinic acid•poly(cytidine-5′-thiophosphoric acid), and

Polyinosinic acid•poly(1-vinylcytidylic acid)

(2) Homopolymer•copolymer Double-stranded RNA

Polyinosinic acid•poly(cytidylic acid, uridylic acid), and

Polyinosinic acid•poly(cytidylic acid, 4-thiouridylic acid).

(3) Copolymer Single-stranded RNA

Poly(adenylic acid, uridylic acid).

(4) Others

5-Fluorouracil, and

Cytosine arabinoside.

Most of single-stranded or double-stranded RNAs among these substances cannot enter into cells directly since they are a polymer and need to be converted into a complex with, for example, a carrier for transferring/introducing a drug into cells. A polynucleotide which formed a complex with a particular drug carrier can enter into a cell. Such a carrier is available in the art or can be prepared according to a method known to one of ordinary skill in the art.

Examples of carriers useful for transferring/introducing a substance into a cell include cationic liposome carriers. Specific examples of cationic liposome carriers include a cationic liposome carrier comprising as basic essential components cationic glycerol and a phospholipid (e.g., phosphatidyl choline, phosphatidyl ethanolamine, phosphatidyl inositol, phosphatidyl serine, lecithin) in the ratio of 1:3 to 2:1 (cationic glycerol:phospholipid) by weight; Lipofectin® (a cationic liposome carrier comprising as basic essential components N-[1-(2,3-dioleyloxy)propyl]-n,n,n-trimethylammonium chloride (DOTMA) and dioleoyl phosphatidylethanolamine (DOPE) in the ratio of 1:1 by weight), Lipofectamine® (a cationic liposome carrier comprising as basic essential components 2,3-dioleyloxy-N-[2-(sperminecarboxamido) ethyl]-N,N-dimethyl-1-propanaminium trifluoroacetate (DOSPA) and dioleoyl phosphatidylethanolamine (DOPE) in the ratio of 3:1 by weight), Cellfectin® (a cationic liposome carrier comprising as basic essential components N, N^{I}, N^{II}, N^{III}-tetramethyl-N, N^{I}, N^{II}, N^{III}-tetrapalmitylspermine (TMTPS) and dioleoyl phosphatidylethanolamine (DOPE) in the ratio of 1:1.5 by weight), DMRIE-C® (a cationic liposome carrier comprising as basic essential components 1,2-dimyristyloxypropyl-3-dimethyl-hydroxyethyl ammonium bromide (DMRIE) and cholesterol in the molar ratio of 1:1). Also included are cationic liposome carriers comprising as basic essential components any one of various glycerol derivatives described in WO94/19314 and a phospholipid (ibid). Among them, a cationic liposome carrier comprising as basic essential components a cationic glycerol and a phospholipid (e.g., lecithin) in the ratio of 1:2 to 1:1 (cationic glycerol:phospholipid) by weight is preferred.

The cationic liposome carrier consists of a lipid bilayer similarly to ordinary liposomes and is an endoplasmic reticulum that is positively charged in aqueous solution. Accordingly, the cationic liposome carrier easily forms a complex with a compound that is negatively charged in aqueous solution such as a nucleic acid.

When the nucleotide used in the present invention is a polynucleotide such as polyinosinic—polycytidylic acid, it can have various chain lengths or molecular weights, and the present invention is by no means restricted to a polynucleotide of any particular chain length or molecular weight. As to a copolymer polynucleotide such as poly(cytidylic acid, uridylic acid), it may contain two nucleotides in various constitutions or ratios, and the present invention is by no means restricted to a polynucleotide of any particular constitution or ratio. However, in the preferred constitution for the polyinosinic acid•poly(cytidylic acid, uridylic acid), one uridylic acid appears as compared with about 12 cytidylic acids (poly C₁₂U). With regard to a copolymer single-stranded RNA such as poly(adenylic acid, uridylic acid), it is preferably a constitution where two nucleotides appear alternately.

The present method is applicable to examination of efficacy of treatment with any types of nucleotide or nucleoside; however, it is suitable for examining or evaluating efficacy of treatment with polyinosinic—polycytidylic acid, and is more suitable for examining efficacy of treatment with polyinosinic—polycytidylic acid of average chain length between 100 bp and 500 bp (base pairs). As mentioned above, polyinosinic—polycytidylic acid cannot enter into a cell directly and should be administered as a complex with a cationic liposome carrier which, for example, comprises as basic essential components a cationic glycerol and a phospholipid (e.g., lecithin) in the ratio of 1:2 to 1:1 (cationic glycerol:phospholipid) by weight so that it can enter into a cell.

The present method can be conducted both in vivo and in vitro. In the former case, examples of tissues of a subject to which a nucleotide or a nucleoside is administered include vein, hepatic artery, portal vein and tumor tissues. In the latter case, examples of tissues to be taken from a subject include a carcinoma tissue and a tissue infected with virus. Specific examples of carcinoma tissues include hepatic carcinoma, lung cancer, carcinoma of colon and rectum, gastric cancer, bladder carcinoma, ovarian carcinoma, uterine cancer, breast cancer, prostatic cancer, brain tumor, kidney cancer, pancreas cancer, skin cancer and myosarcoma. However, the present invention is not limited to the above tissues, and one can select tissues appropriately depending on the purpose.

According to the present method, a nucleotide or a nucleoside is generally administered as a solution. The nucleotide or nucleoside can be in any form before being converted into a solution, including liquid preparation, powder preparation and lyophilized preparation. The present invention is not restricted to a particular form.

In the present examination method, any of known pharmaceutical compositions containing a nucleotide or a nucleoside that can enter into cells can be used as it is, which composition comprises, for example, a complex between a double stranded RNA such as polyinosinic—polycytidylic acid and a cationic liposome carrier. Such a pharmaceutical composition can be prepared in a conventional manner or are commercially available.

The dose of a nucleotide or a nucleoside should vary depending on the kinds of nucleotide or nucleoside to be used, the disease, conditions of the patient, or the like, and is appropriately determined.

III. Determination of Expression Amount of Protein

In the present examination method, the determination of the expression amount of a protein according to the present invention can be conducted by a known method wherein (1) the target protein is measured directly, or (2) the expression of mRNA encoding the target protein is measured, and the like. Specific examples of the former include ELISA method that uses an appropriate antibody and specific examples of the latter include RT-PCR method.

When carrying out the present method, the expression amount of a protein according to the present invention is measured in vivo or in vitro, before and after the administration of a nucleotide or a nucleoside into a tissue of a subject or into a tissue obtained from the subject. The increase in the amount of expression of the protein as a result of administration of the nucleotide or the nucleoside leads to the evaluation that a pharmaceutical composition containing the nucleotide or the nucleoside possibly has therapeutic effect in said patient.

Among the proteins according to the present invention, preferred protein of which expression amount is measured varies depending on the kind of nucleotide or nucleoside to be used, the kind of disease (e.g., tumor, infected virus, etc.), conditions of the patient, and the like. To examine therapeutic efficacy of a pharmaceutical composition containing as a double-stranded RNA polyinosinic—polycytidylic acid and cationic liposome carrier, it would be useful to measure the protein having amino acid sequence of SEQ ID NO: 4. When the expression amount of the protein of SEQ ID NO: 4 increases after the administration of said pharmaceutical composition, said composition can be estimated to have therapeutic effect in said patient.

The criterion of judgment regarding therapeutic efficacy should vary depending on the condition of a patient, kind of nucleotide or nucleoside to be used, the kind of disease (e.g., tumor, infected virus, etc.), a protein to be measured, method of measurement, and the like. However, if the expression amount of a protein measured after the administration increased 1.5 or more times, preferably, 2.0 or more times as compared to what measured before the administration, it can be estimated to be therapeutically effective.

IV. Determination of Activity of Protein

In the present examination method, the determination of the activity of a protein according to the present invention can be conducted by a known method. Examples of such activity include nuclease (DNase, RNase) activity, kinase activity and protein-binding activity. Specific examples of the method for determining the activity involve the determination of nuclease (DNase, RNase) activity or kinase activity as hereinafter described.

(1) Determination of DNase Activity

Substrate:

Cell fraction, DNA or DNA plasmid, which are radio-labeled with a radioisotope such as radioactive phosphorous

Reaction Solution:

50 mM MES-NaOH(pH 5.6) or 10 mM Tris-HCl (pH 7.8), 3 mM CaCl₂, 3 mM MgCl₂, 0.1 mM PMSF, 1 mM mercaptoethanol

To the substrate and the reaction solution is added a test protein and the mixture is incubated at 37° C. for 10 to 60 minutes. The reaction mixture is centrifuged at 20,000×g for 20 seconds and the supernatant is removed. The precipitates are treated with RNase and proteinase K, and electrophoresed on agarose. The DNase activity can be determined from the amount of decomposed radio-labeled DNA as an indicator.

(2) Determination of RNase Activity

Substrate:

Ribosomal RNA radio-labeled with a radioisotope such as radioactive phosphorous

Reaction Solution:

10 mM Tris-HCl(pH 7.4), 300 mM NaCl, 5 mM EDTA

To the substrate and the reaction solution is added a test protein and the mixture is incubated at 37° C. for 10 to 60 minutes. The radio-labeled ribosomal RNA is extracted by guanidium thiocyanate method and electrophoresed on agarose. The RNase activity can be determined from the amount of fragmented radio-labeled RNA as an indicator.

(3) Determination of Kinase Activity

Substrate:Peptide

Reaction solution:

20-50 mM Tris-HCl (pH 7.4), 5-10 mM MgCl₂, 250-500 μM [γ-³²P]ATP, 15 mM mercaptoethanol, 0.1% Tween80

To the substrate and the reaction solution is added a test protein and the mixture is incubated at 37° C. for 10 to 60 minutes. The substrate peptides are extracted and the kinase activity can be determined from the amount of radio-labeled peptides as an indicator.

When carrying out the present method, the nuclease activity or the like is measured in vivo or in vitro, before and after the administration of a nucleotide or a nucleoside into a tissue of a subject or into a tissue obtained from the subject. The increase in the activity as a result of administration of the nucleotide or the nucleoside leads to the evaluation that a pharmaceutical composition containing the said nucleotide or the nucleoside possibly has therapeutic effect in said patient.

Among the proteins according to the present invention, preferred protein of which expression amount should be measured varies depending on the kind of nucleotide or nucleoside to be used, the kind of disease (e.g., tumor, infected virus, etc.), conditions of the patient, and the like. To examine therapeutic efficacy of a pharmaceutical composition containing as a double-stranded RNA polyinosinic—polycytidylic acid and cationic liposome carrier, it would be useful to measure a protein from those having the amino acid sequences of SEQ ID NO: 1-4, preferably SEQ ID NO: 1, 2 and 4. When the expression amount of a protein having an amino acid sequence selected from SEQ ID NO: 1-4 increases after the administration of said pharmaceutical composition, said composition can be estimated as being possibly therapeutically effective in said patient.

The criterion of judgment regarding therapeutic efficacy varies depending on the kind of nucleotide or nucleoside to be used, condition of a patient, the kind of disease (e.g., tumor, infected virus, etc.), a protein to be measured, method of measurement, and the like. However, if the activity of a protein measured increased 1.5 or more times, preferably, 2.0 or more times as compared to what measured before the administration, it can be estimated as being therapeutically effective.

V. Determination of Binding Ability to Proteins

Examples of method for examining the binding ability of a test substance (medical substance) to a protein according to the present invention include West western method with an appropriate antibody, immunoprecipitation method, and a process by means of BIAcore® (Pharmacia Biotech) which is an automated analytic device based on surface plasmon resonance (SPR), an optical phenomenon, as measuring principle. If a test substance proved to bind to a protein(s) according to the present invention, said test substance can be a promising antitumor or antivirus agent.

A substance known to have antitumor or antivirus activity can also be examined for the ability to bind to a protein of the present invention according to a similar method as the above. If the substance proved to bind to the protein, said substance can be expected to act in vivo through at least a similar reaction mechanism as polyinosinic—polycytidylic acid.

The experimental examples below provides the detection or identification of a protein(s) according to the present invention, and influence of a nucleic acid on the expression of mRNA for the protein. In the following experimental examples, a double-stranded RNA, polyinosinic acid-polycytidylic acid (polyinosinic—polycytidylic acid, hereinafter, referred to as “poly(I)•poly(C)”) was used as a nucleic acid-containing test substance (drug). Poly(I)•poly(C) is known to be efficacious against certain cancer cells (e.g., HeLaS3, A431) but inefficacious against others (e.g., HeLa, SW620) (Cancer Research, 59, pp. 4325-4333 (1999)).

### EXPERIMENTAL EXAMPLE 1

Detection of Binding Ability to Proteins of SEQ ID NO: 1-3

(1) Each protein was synthesized by plating phages containing cDNA on a petri dish using human HeLa 5′-STRETCH PLUS cDNA library.

(2) The synthesized protein was transferred to nitrocellulose membrane.

(3) To the nitrocellulose membrane was added the following test solution containing poly(I)•poly(C) (chain length 100-500 bp) radio-labeled with [γ-³²P]ATP, and subjected to hybridization at 42° C., for 5-6 hours.

<Test Solution>

10 mM HEPES, pH 7.5

50 mM NaCl

1 mM Dithiothreitol

1 mM EDTA

5% Glycerol

1 ng/ml-1 μg/ml [³²P]-poly(I)•poly(C)

(4) The nitrocellulose membrane was washed several times with above-mentioned solution which does not contain [³²P]-poly(I)•poly(C) and exposed to imaging plate to screen proteins bound to poly(I)•poly(C). The nucleotide sequence of cDNA corresponding to the screened protein was determined with a sequencer to identify the screened protein.

As a result, proteins of SEQ ID NO: 1-3 were revealed to that bound to poly(I)•poly(C).

### EXPERIMENTAL EXAMPLE 2

Detection of Expression of mRNA for Protein of SEQ ID NO: 4 (ISG20)

(1) A test complex comprising poly(I)•poly(C) was added to each of cancer cells, Hela, HeLaS3, A431 and SW620 cells so that the concentration of poly(I)•poly(C) becomes from 10 to 100 ng/ml.

(2) Following the addition of the test complex, total RNA was extracted from HeLa and HeLaS3 cells at 2 and 4 hours, and from A431 and SW620 cells at 0.5, 1, 2 and 4 hours by guanidium thiocyanate method.

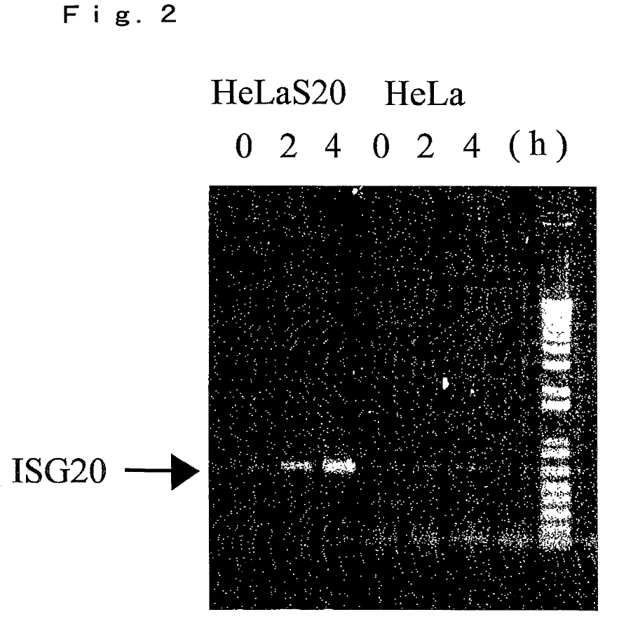

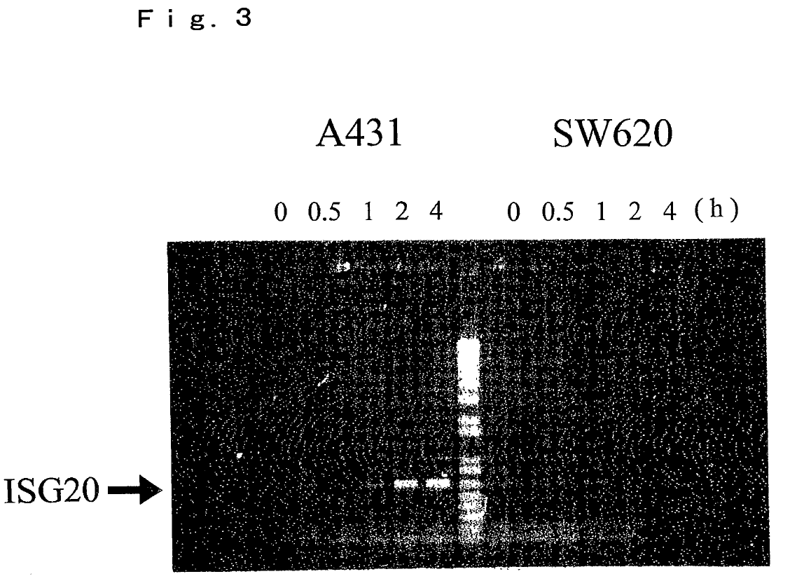

(3) The extracted RNA was subjected to the Reverse Transcriptase Chain Reaction (RT-PCR) and the reaction solution was electrophoresed on agarose gel. The bands indicated by white arrow in FIGS. 2 and 3 show ISG20 mRNA.

<Test Complex>

| | Weight ratio |
|---|---|
| Poly (I) · poly (C) (chain length 100˜500 bp) | 1 |
| Cationic glycerol | 6 |
| Yolk lecithin | 10 |

As is clear from FIGS. 2 and 3, the expression amount of ISG20 mRNA increased with time in cancer cells (HeLaS3, A431) on which the complex comprising poly(I)•poly(C) has effect. On the other hand, ISG20 mRNA was not expressed in cancer cells (HeLa, SW620) on which the complex comprising poly(I)•poly(C) does not have effect. This result shows that it is possible to evaluate whether a test drug is effective as a therapeutic agent in a specific patient on the basis of the presence or absence of expression of ISG20.

### INDUSTRIAL APPLICABILITY

According to the method of the present invention, the efficacy of an antitumor or antivirus agent comprising a nucleic acid in individual patients can be evaluated beforehand, and hence it becomes possible to treat a patient more effectively through tailor-made therapy or the like. Furthermore, according to the present method, a substance potentially having antitumor or antivirus activity can be screened.

1. A method of examining efficacy of treatment with a nucleotide or a nucleoside in a given subject comprising:
(a) measuring the amount of expression or activity of at least one protein among those each having amino acid sequences of SEQ ID NO: 1-4 in a blood or tissue sample previously taken from the subject;
(b) administering the nucleotide or the nucleoside into a tissue of the subject or a tissue taken from the subject followed by measuring the amount of expression or activity of the same protein as that measured in (a) in blood or treated tissue after 3-48 hours from administration; and
(c) comparing the measurement obtained in step (a) with that obtained in step (b) and evaluating the change in the amount of expression or the activity of the protein.

2. A method of examining efficacy of treatment with a nucleotide or a nucleoside in a given subject comprising:
(a) measuring the amount of expression or activity of at least one protein among those each having amino acid sequences of SEQ ID NO: 5-30 in a blood or tissue sample previously taken from the subject;
(b) administering the nucleotide or the nucleoside into a tissue of the subject or a tissue taken from the subject followed by measuring the amount of expression or activity of the same protein as that measured in (a) in blood or treated tissue after 3-48 hours from administration; and
(c) comparing the measurement obtained in step (a) with that obtained in step (b) and evaluating the change in the amount of expression or the activity of the protein.

3. The method of claim 1 or 2, wherein the nucleotide or nucleoside exerts antitumor or antivirus activity at least when entered into a cell and is used in the form of a complex with a carrier effective to transfer the same into a cell.

4. The method of claim 3, wherein the carrier is cationic liposome carrier.

5. The method of claim 4, wherein the cationic liposome carrier is a cationic liposome carrier comprising as basic essential components 2-O-(2-diethylaminoethyl)carbamoyl-1,3-O-dioleoyl glycerol and a phospholipid in the ratio of 1:3 to 2:1 (the glycerol derivative:the phospholipid) by weight; Lipofectin® (a cationic liposome carrier comprising as basic essential components N-[1-(2,3-dioleyloxy)propyl]-n,n,n-trimethylammonium chloride (DOTMA) and dioleoyl phosphatidylethanolamine (DOPE) in the ratio of 1:1 by weight), Lipofectamine® (a cationic liposome carrier comprising as basic essential components 2,3-dioleyloxy-N-[2-(sperminecarboxamido) ethyl]-N,N-dimethyl-1-propanammonium trifluoroacetate (DOSPA) and dioleoyl phosphatidylethanolamine (DOPE) in the ratio of 3:1 by weight), Cellfectin® (a cationic liposome carrier comprising as basic essential components N, N^{I}, N^{II}, N^{III}-tetramethyl-N, N^{I}, N^{II}, N^{III}-tetrapalmitylspermine (TMTPS) and dioleoyl phosphatidylethanolamine (DOPE) in the ratio of 1:1.5 by weight), or DMRIE-C® (a cationic liposome carrier comprising as basic essential components 1,2-dimyristyloxypropyl-3-dimethyl-hydroxyethyl ammonium bromide (DMRIE) and cholesterol in the molar ratio of 1:1).

6. The method of claim 1 or 2, wherein the nucleotide or the nucleoside is a homopolymer•homopolymer double-stranded RNA, homopolymer•copolymer double-stranded RNA, copolymer single-stranded RNA, 5-Fluorouracil, or cytosine arabinoside.

7. The method of claim 6, wherein the homopolymer•homopolymer double-stranded RNA is selected from the group consisting of polyinosinic acid•polycytidylic acid, polyinosinic acid•poly(5-bromocytidylic acid), polyinosinic acid•poly(2-thiocytidylic acid), poly(7-deazainosinic acid)•polycytidylic acid, poly(7-deazainosinic acid)•poly(5-bromocytidylic acid), poly(2′-azidoinosinic acid)•polycytidylic acid, polyinosinic acid•poly(cytidine-5′-thiophosphoric acid), and polyinosinic acid poly(1-vinylcytidylic acid).

8. The method of claim 6, wherein the homopolymer• copolymer double-stranded RNA is polyinosinic acid•poly(cytidylic acid, uridylic acid) or polyinosinic acid•poly(cytidylic acid, 4-thiouridylic acid).

9. The method of claim 6, wherein the copolymer single-stranded RNA is poly(adenylic acid, uridylic acid).

10. A method of examining efficacy of treatment with a complex of polyinosinic acid•polycytidylic acid of average chain length between 100 bp and 500 bp with a cationic liposome carrier which comprises as basic essential components 2-O-(2-diethylaminoethyl)carbamoyl-1,3-O-dioleoyl glycerol and a phospholipid in the ratio of 1:3 to 2:1 (the glycerol derivative:the phospholipid) by weight in a given subject comprising:
(a) measuring the amount of expression or activity of at least one protein among those each having amino acid sequences of SEQ ID NO: 1-4 in a blood or tissue sample previously taken from the subject;
(b) administering the nucleotide or the nucleoside into a tissue of the subject or a tissue taken from the subject followed by measuring the amount of expression or activity of the same protein as that measured in (a) in blood or treated tissue after 3-48 hours from administration; and
(c) comparing the measurement obtained in step (a) with that obtained in step (b) and evaluating the change in the amount of expression or the activity of the protein.

11. A method of screening for agents potentially having antitumor or antivirus activity comprising examining a test compound for ability to bind to at least one protein among those each having amino acid sequences of SEQ ID NO: 1-4.

12. A method of screening for agents potentially having antitumor or antivirus activity comprising examining a test compound for ability to bind to at least one protein among those each having amino acid sequences of SEQ ID NO: 5-30.

13. A method of investigating the mechanism of action of an antitumor or antivirus agent in vivo comprising examining the antitumor or antivirus agent for ability to bind to at least one protein among those each having amino acid sequences of SEQ ID NO: 1-4.

14. A method of investigating the mechanism of action of an antitumor or antivirus agent in vivo comprising examining the antitumor or antivirus agent for ability to bind to at least one protein among those each having amino acid sequences of SEQ ID NO: 5-30.
